(19) United States
(12) Patent Application Publication
(10) Pub. No.: US 2007/0068522 A1
(43) Pub. Date: Mar. 29, 2007

(54) Nebulizer device for application to small beds, sun beds, deckchairs or the like

(76) Inventor: Giovanni Colombo, Cantu (CO) (IT)

Correspondence Address: HEDMAN & COSTIGAN P.C., 1185 AVENUE OF THE AMERICAS, NEW YORK, NY, 10036, US

(21) Appl. No.: 11/500,818

(22) Filed: Aug. 8, 2006

(30) Foreign Application Priority Data
Sep. 29, 2005 (IT) MI2005A 001828

(51) Int. Cl. A61M 15/00; A61M 16/10

(52) U.S. Cl. 128/203.12

(57) ABSTRACT

A nebulizer device for application to small beds, sun beds, deckchairs or the like comprises tank means adapted to supply nebulizer means which can be coupled to the bed construction and can be driven by an user through pump means; the nebulizer and pump means being adapted to be coupled to the bed construction, the deckchair or the like, through quick coupling attachment means.

### BACKGROUND OF THE INVENTION

The present invention relates to a nebulizer device which has been specifically designed for application to small beds, sun beds or deckchairs.

As is known, a broad diffused practice is that of taking a sunbath on small beds or deckchairs on a beach or a solarium.

To that end, a lot of different small bed and deckchair types have been designed, which, while they provide a satisfactory support function, are not adapted to solve some problems related to the sun ray exposure thereby the user can feel an excessive heat sensation and can desire to refresh before continuing his/her exposure to the sun rays.

On the other hand, this requirement can be difficult to be met for a lot of different reasons.

For example, for health reasons, it is not possible to take a full bath. Moreover, the beach bathing plants may be arranged at a rather remote place.

Moreover, for taking a bath or shower, the user must interrupt for several minutes his/her exposure to the sun or, in a solarium, to UV lamps, the cost of which would be anyhow charged to the client.

This difficulty or inconvenience to wet his/her body parts, i.e. the body parts exposed to UV and/or IR radiations, inevitably causes a longer tanning time.

In fact, the stifling heat tends to cause the person to interrupt his/her exposure to the UV or IR or sun rays.

Moreover, a dry skins hinders a proper tanning, with respect to a wet skin, since the water droplets spread on the skin provide a so-called lens effect concentrating the impinging radiation and enhancing its tanning efficiency.

### SUMMARY OF THE INVENTION

Accordingly, the aim of the present invention is to provide such a device adapted to overcome the above mentioned drawbacks.

Within the scope of the above mentioned aim, a main object of the present invention is to provide such a device which can be easily applied to any types of small beds or deckchairs as commercially available.

Another object of the present invention is to provide such a device which is very simple construction wise and, moreover, which can be easily operated.

Yet another object of the present invention is to provide such a device which can be made at a low cost and which, moreover, is very reliable in operation and easily serviced.

According to one aspect of the present invention, the above mentioned aim and objects, as well as yet other objects, which will become more apparent hereinafter, are achieved by a nebulizer device for application to small beds, sun beds or deckchairs, characterized in that said nebulizer device comprises tank means, designed for supplying nebulizer means adapted to be coupled to the small bed construction and to be operated by a user through pump means, at least said nebulizer means and pump means being adapted to be coupled to the construction of said small bed, sun bed, deckchair or the like, through quick attachment coupling means.

### BRIEF DESCRIPTION OF THE DRAWINGS

Further characteristics and advantages of the present invention will become more apparent herein-after from the following detailed disclosure of a preferred, though not exclusive, embodiment of the invention which is illustrated, by way of an indicative, but not limitative, example in the accompanying drawings, where:

FIG. 1 is a perspective view showing the main component elements of the nebulizer device according to the present invention;

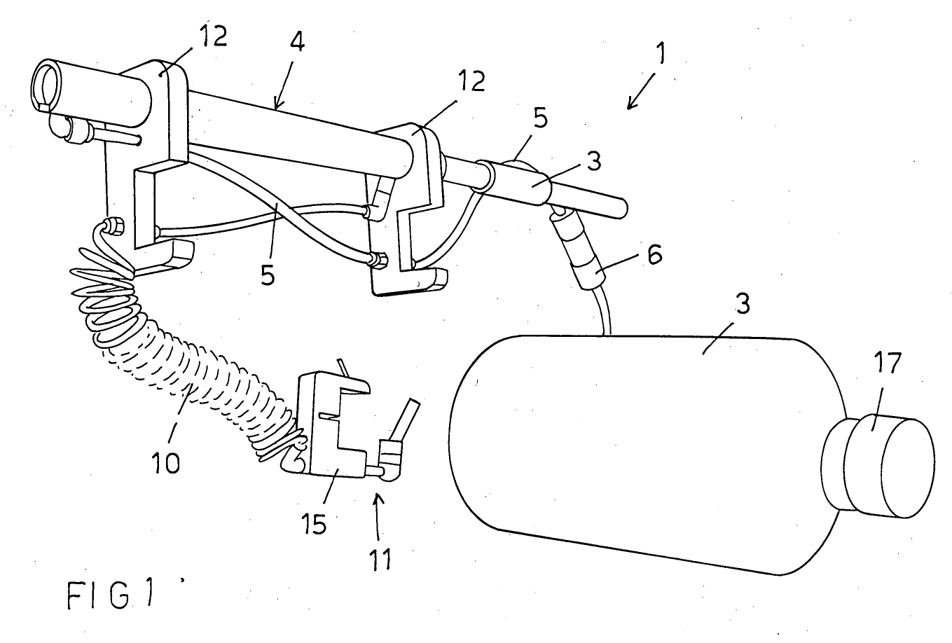

FIG. 2 is a perspective view of a small bed including the nebulizer device according to the invention; and

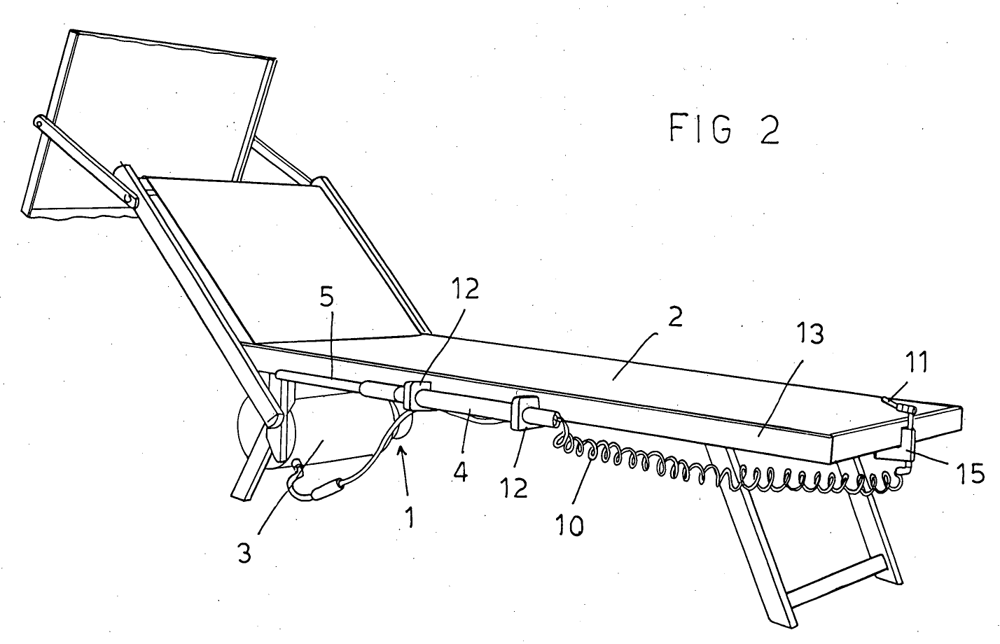

FIG. 3 is a view similar to FIG. 2, and showing the use of the nebulizing device according to the present invention.

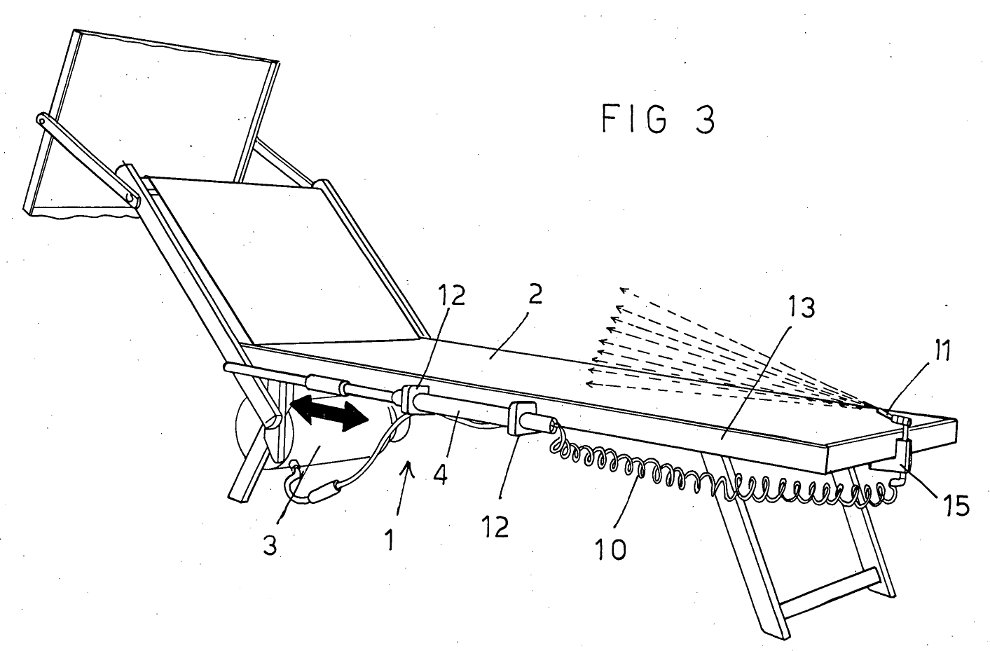

### DESCRIPTION OF THE PREFERRED EMBODIMENTS

With reference to the number references of the above mentioned figures, the nebulizer or nebulizing device according to the present invention, which has been generally indicated by the reference number 1, comprises a tank 3, which can be advantageously thermally insulated, and adapted to be filled-in by liquids in general, said tank including a flexible connector 6 coupled, through a flexible duct 5, to an end portion of a pump 4 including a rod element 3 operating as a manual control element.

Alternately, the pump 4 can comprise an electric pump, advantageously a battery operated electric pump, due to safety and easy operation reasons, and it will be switched on and off through a manually operated switch element, not specifically shown.

The nebulizer device according to the invention can also optionally comprise timed detecting or sensor elements.

As shown, the pump 4 is coupled, downstream thereof, to a flexible delivery hose 10, conveying the liquid supplied by said pump 4 to an ejector or nebulizer 11, the flow of which can be easily adjusted in rate and direction in either a manual or electrical manner, and which is mechanically coupled to the base or bottom portion of the bed.

The tank 3, in turn, can be easily coupled by any desired suitable coupling means or a pair of belts, wound under or on the bottom of the tank 3 and having an end portion thereof fixedly coupled to the bottom of the bed construction 2, or other supporting element the device 1 is applied to.

Advantageously, the pump 4 is coupled to a pair of brackets, respectively indicated by the reference numbers 12, and adapted to be easily applied to the longitudinal elements or beams 13 of the small bed 2.

The nebulizer 11 is also advantageously mounted on a bracket 15, which can be easily applied to any desired element of the bed, at a desired position, owing to the provision of the flexible delivery hose 10.

In this connection it should be apparent that it would be also possible to provide a plurality of nebulizers, and not only one as in the shown embodiment.

In such a case, a plurality of pumps and/or switches can also be provided, of a corresponding plurality of timed detectors or sensors, as well as delivery flexible hoses, to provide the possibility to easily choose an ejector set for nebulizing a given region.

In all the provided embodiments, it is advantageously possible to also provide wind barriers, preferably made of a clear plexiglas material, and being coupled to the element supporting the nebulizer device according to the invention.

That barriers would be very useful as the subject device is used outside, to stabilize, under a comparatively strong wind, the direction of the jet ejected by the ejector elements.

It is to be pointed out that the shape and size of the elements constituting the subject liquid ejector device, which has been specifically designed for application to small beds, deckchairs and the like, can be varied according to requirements, while falling within the scope of the invention as above disclosed.

The operation of the device according to the invention can be easily deduced from the above disclosure.

In particular, after having filled-in water into the tank 3, for example through the attachment 17, the user, lying down on the bed 2, will operate at will the rod 5 of the pump 4, to cause water to be ejected by the nebulizer element 11.

It has been found that the invention fully achieves the intended aim and objects.

In fact, the invention provides a nebulizer device which can be easily applied to small beds, sun beds, deckchairs and the like, and having the following advantages.

Since it is possible to easily wet the most delicate parts of the human body by wetting them, the heat can be easily sustained during the sun or UV-IR lamp exposure.

A further advantage is that the user can wet his/her body in a stationary condition.

Moreover, since the nebulizer device according to the invention can be easily controlled, it can be also used by partially disabled persons.

Furthermore, the nebulizer device according to the invention can be easily installed, and it does not require any replacements of the existing bed or deckchairs, but it merely requires very simple assembling operations, and, moreover, it has a very low cost.

The desired tanning can be achieved in a quicker time and in a much more even manner, since a wet skin, as above mentioned, causes the UV radiation to efficiently operate on the human body.

Finally, a plurality of nebulizer device according to the invention can be used in a bathing establishment or in a solarium, operated by a single operator, and, consequently, with very low managing costs.

In practicing the invention, the used materials, as well as the contingent size and shapes, can be any, according to requirements.

1. A nebulizer device for application to small beds, sun beds or deckchairs, characterized in that said nebulizer device comprises tank means, designed for supplying nebulizer means adapted to be coupled to the small bed construction and to be operated by a user through pump means, at least said nebulizer means and pump means being adapted to be coupled to the construction of said small bed, sun bed, deckchair or the like, through quick attachment coupling means.

2. A nebulizer device, according to claim 1, characterized in that said tank means are thermally insulated and adapted to be filled-in by liquids, said tank means comprising a flexible connector coupled, through a flexible hose, to one end portion of a pump including a manual control constituted by a control rod.

3. A nebulizer device, according to claim 1, characterized in that said pump is coupled, downstream thereof, to a delivery flexible hose, conveying the liquid provided by said pump to an ejector or nebulizer element, having an outlet flow which can be adjusted in rate and direction, either manually or electrically, said ejector or nebulizer element being mechanically coupled to a base of said small bed.

4. A nebulizer device, according to claim 1, characterized in that said tank means are coupled to said small bed by suitable coupling means or a pair of coupling belts, wound under said tank means and having an end portion thereof fixedly coupled to the bottom of the bed construction, or other element, to which said nebulizer device is applied.

5. A nebulizer device, according to claim 1, characterized in that said pump is coupled to a pair of brackets in turn coupled to longitudinal beams of said small bed.

6. A nebulizer device, according to claim 1, characterized in that said nebulizer element is mounted on a bracket applied to an element of said small bed, at a set position, owing to the provision of said flexible delivery hose.

7. A nebulizer device, according to claim 1, characterized in that said nebulizer device comprises a plurality of nebulizer elements.

8. A nebulizer device, according to claim 1, characterized in that said nebulizer device comprises a plurality of pumps and/or switches, and of flexible delivery hose.

9. A nebulizer device, according to claim 1, characterized in that said pump is an electrical battery operated pump including a manual operation switch.

10. A nebulizer device, according to claim 1, characterized in that it comprises a plurality of timed detector or sensor means.
